(19) United States
(12) Patent Application Publication
(10) Pub. No.: US 2007/0293844 A1
(43) Pub. Date: Dec. 20, 2007

(54) INTRAOCULAR PRESSURE CONTROL

(76) Inventors: Nader Nazarifar, Laguna Niguel, CA (US); Frederick Reed, Cypress, CA (US); John C. Huculak, Mission Viejo, CA (US); Roger Thomas, Tustin, CA (US); Steven T. Charles, Memphis, TN (US)

Correspondence Address: ALCON, IP LEGAL, TB4-8, 6201 SOUTH FREEWAY, FORT WORTH, TX, 76134, US

(21) Appl. No.: 11/829,333

(22) Filed: Jul. 27, 2007

Related U.S. Application Data
(63) Continuation-in-part of application No. 11/237,503, filed on Sep. 28, 2005.

(51) Int. Cl. A61M 5/14

(52) U.S. Cl. 604/505

(57) ABSTRACT

An improved method of controlling intraocular pressure with a microsurgical system using measured flow rate and a dual infusion chamber.

This application is a continuation-in-part of U.S. application Ser. No. 11/237,503 filed Sep. 28, 2005.

### FIELD OF THE INVENTION

The present invention generally pertains to microsurgical systems and more particularly to controlling intraocular pressure in ophthalmic surgery.

Description of the Related Art

During small incision surgery, and particularly during ophthalmic surgery, small probes are inserted into the operative site to cut, remove, or otherwise manipulate tissue. During these surgical procedures, fluid is typically infused into the eye, and the infusion fluid and tissue are aspirated from the surgical site.

Maintaining an optimum intraocular pressure during ophthalmic surgery is currently problematic. When no aspiration is occurring, the pressure in the eye becomes the pressure of the fluid being infused into the eye. This pressure is typically referred to as the “dead head pressure”. However, when aspiration is applied, the intraocular pressure drops dramatically from the dead head pressure due to all the pressure losses in the aspiration circuit associated with aspiration flow. Therefore, ophthalmic surgeons currently tolerate higher than desired dead head pressures to compensate for occasions when aspiration would otherwise lower the intraocular pressure to soft-eye conditions. Clinically, such over-pressurizing of the eye is not ideal.

Accordingly, a need continues to exist for an improved method of controlling intraocular pressure during ophthalmic surgery.

### SUMMARY OF THE INVENTION

In one aspect, the present invention is a method of controlling intraocular pressure with a microsurgical system. An infusion chamber containing an irrigating fluid is provided, and a desired intraocular pressure is selected. The infusion chamber is pressurized with a pressurized gas to provide irrigating fluid to a surgical device. A second fluid is aspirated from an eye with the device. A flow rate of the irrigating fluid within a fluid line fluidly coupled to the surgical device is measured. A signal corresponding to the measured flow rate is provided to a computer. A predicted intraocular pressure is calculated with the computer in response to the signal. A level of the pressurized gas is adjusted in response to a second signal from the computer to maintain the predicted intraocular pressure proximate the desired intraocular pressure. The pressurizing of the infusion chamber is ceased in response to the surgical device ceasing to aspirate the second fluid from the eye.

### BRIEF DESCRIPTION OF THE DRAWINGS

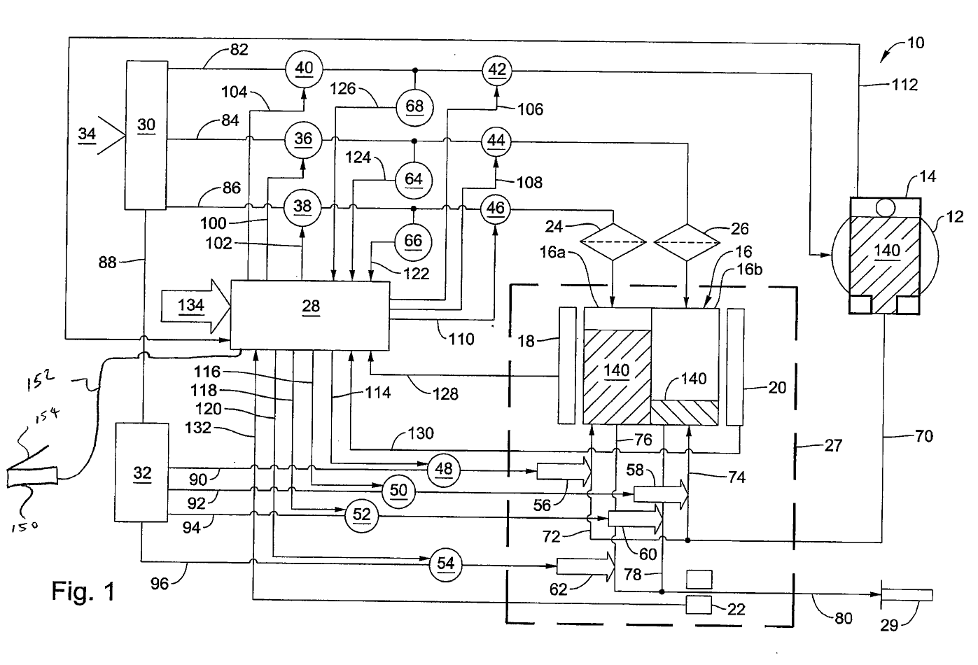

For a more complete understanding of the present invention, and for further objects and advantages thereof, reference is made to the following description taken in conjunction with the accompanying drawing, in which FIG. 1 is a schematic diagram illustrating infusion control in an ophthalmic microsurgical system.

### DETAILED DESCRIPTION OF THE PREFERRED EMBODIMENTS

The preferred embodiment of the present invention and their advantages are best understood by referring to FIG. 1 of the drawings. As shown in FIG. 1, ophthalmic microsurgical system 10 includes a pressure cuff 12; an infusion source 14; a dual infusion chamber 16 having a chamber 16a and a chamber 16b; fluid level sensors 18 and 20; a flow sensor 22; filters 24 and 26; a surgical device 29; a computer or microprocessor 28; gas manifolds 30 and 32; a pressurized gas source 34; proportional solenoid valves 36, 38, and 40; “on/off” solenoid valves 42, 44, 46, 48, 50, 52, 54; actuators 56, 58, 60, and 62; and pressure transducers 64, 66, and 68. Dual infusion chamber 16; fluid level sensors 18 and 20; portions of infusion fluid lines 70, 72, 74, 76, 78, and 80; and portions of gas lines 84 and 86 are preferably disposed in a surgical cassette 27. Infusion source 14; dual infusion chamber 16; flow sensor 22; filters 24 and 26; and surgical device 29 are fluidly coupled via infusion fluid lines 70-80. Infusion source 14, dual infusion chamber 16, gas manifolds 30 and 32; pressurized gas source 34; and actuators 56, 58, 60, and 62 are fluidly coupled via gas lines 82, 84, 86, 88, 90, 92, 94, and 96. Infusion source 14; fluid level sensors 18-20; flow sensor 22; microprocessor 28; proportional solenoid valves 36-40; on/off solenoid valves 42-54; actuators 56-62; and pressure transducers 64-68 are electrically coupled via interfaces 100, 102, 104, 106, 108, 110, 112, 114, 116, 118, 120, 122, 124, 126, 128, 130, and 132.

Infusion source 14 is preferably a flexible infusion source. Fluid level sensors 18 and 20 may be any suitable device for measuring the level of fluid in infusion chambers 16a and 16b, respectively. Fluid level sensors 18 and 20 are preferably capable of measuring the level of fluid in infusion chambers 16a and 16b in a continuous manner. Flow sensor 22 may be any suitable device for measuring the flow rate of fluid within fluid line 80. Flow sensor 22 is preferably a non-invasive flow sensor. Filters 24 and 26 are hydrophobic micro-bacterial filters. A preferred filter is the Versapor® membrane filter (0.8 micron) available from Pall Corporation of East Hills, N.Y. Microprocessor 28 is capable of implementing feedback control, and preferably PID control. Surgical device 29 may be any suitable device for providing surgical irrigating fluid to the eye but is preferably an infusion cannula, an irrigation handpiece, or and irrigation/aspiration handpiece.

In operation, fluid lines 70, 72, and 74; chambers 16a and 16b; fluid lines 76, 78, and 80; and surgical device 29 are all primed with a surgical irrigating fluid 140 by pressurizing infusion source 14. Surgical irrigating fluid 140 may be any surgical irrigating fluid suitable for ophthalmic use, such as, by way of example, BSS PLUS® intraocular irrigating solution available from Alcon Laboratories, Inc.

The pressurizing of infusion source 14 is preferably performed by pressure cuff 12. More specifically, microprocessor 28 sends a control signal to open solenoid valve 42 via interface 106 and to close solenoid valves 44 and 46 via interfaces 108 and 110, respectively. Microprocessor 28 also sends a control signal to open proportional solenoid valve 40 via interface 104 so that manifold 30 supplies the appropriate amount of pressurized air to actuate pressure cuff 12. Pressure transducer 68 senses the pressure within gas line 82 and provides a corresponding signal to microprocessor 28 via interface 126. Solenoid valves 48-54 are initially open so that manifold 32 provides pressurized air to actuate actuators 56-62 to close fluid lines 72-78. Microprocessor 28 sends control signals to close solenoid valves 48-54 via interfaces 114-120. The closing of solenoid valves 48-54 actuates actuators 56-62 to open fluid lines 72-78. After all chambers and fluid lines are primed, microprocessor 28 closes actuators 56-62 and thus fluid lines 72-78. Alternatively, the pressuring of infusion source 14 may be performed solely via gravity.

After priming, a user then provides a desired intraocular pressure to microprocessor 28 via an input 134. Input 134 may be any suitable input device but is preferably a touch screen display or physical knob. Chamber 16b is preferably the initial active infusion chamber. Microprocessor 28 sends appropriate control signals to open solenoid valve 44 and to open proportional solenoid valve 36 (via interface 100) to provide an appropriate level of pressurized air to chamber 16b. Pressure transducer 64 senses the pressure within gas line 84 and provides a corresponding signal to microprocessor 28 via interface 124. Microprocessor 28 also sends an appropriate control signal to open actuator 60 and thus fluid line 78. Chamber 16b supplies pressurized fluid 140 to the eye via fluid lines 78 and 80 and surgical device 29. Flow sensor 22 measures the flow rate of fluid 140 and provides a corresponding signal to microprocessor 28 via interface 132. Microprocessor 28 calculates a predicted intraocular pressure using the signal from flow sensor 22 and empirically determined impedance information of microsurgical system 10. Microprocessor 28 then sends an appropriate feedback control signal to proportional solenoid valve 36 to maintain the predicted intraocular pressure at or near the desired intraocular pressure during all portions of the surgery.

Fluid level sensor 20 continuously monitors the decrease in the level of fluid 140 in chamber 16b during surgery and provides a corresponding signal to microprocessor 28 via interface 130. Microprocessor 28 performs adjustments to the air pressure provided to chamber 16b to accommodate for the difference in fluid head height as the level of fluid 140 decreases. When the level of fluid 140 in chamber 16b reaches a bottom limit level, microprocessor 28 closes solenoid valve 44 and actuator 60 and opens solenoid valve 46 and actuators 58 and 62. Chamber 16a is now the active infusion chamber. Microprocessor 28 sends an appropriate control signal to proportional solenoid valve 38 via interface 102 to provide an appropriate level of pressurized air to chamber 16a. Pressure transducer 66 senses the pressure within gas line 86 and provides a corresponding signal to microprocessor 28 via interface 122. Chamber 16a supplies pressurized fluid 140 to the eye via fluid lines 76 and 80 and surgical device 29. Flow sensor 22 measures the flow rate of fluid 140 and provides a corresponding signal to microprocessor 28 via interface 132. Microprocessor 28 calculates the predicted intraocular pressure as described above and the sends an appropriate feedback signal to proportional solenoid valve 38 to maintain the predicted intraocular pressure at or near the desired intraocular pressure during all portions of the surgery. Microprocessor 28 closes actuator 58 and fluid line 74 once chamber 16b is refilled with fluid 140.

Fluid level sensor 18 continuously monitors the decrease in the level of fluid 140 in chamber 16a during surgery and provides a corresponding signal to microprocessor 28 via interface 128. Microprocessor 28 performs adjustments to the air pressure provided to chamber 16a to accommodate for the difference in fluid head height as the level of fluid 140 decreases. When the level of fluid 140 in chamber 16a reaches a bottom limit level, microprocessor 28 switches chamber 16b to active infusion, makes chamber 16a inactive, and refills chamber 16a with fluid 140 via fluid line 72. This cycling between chambers 16b and 16a continues throughout the surgery.

Infusion source 14 is preferably monitored via a fluid level sensor (not shown) capable of providing a signal to microprocessor 28 via interface 112 when source 14 reaches a near empty limit. Chambers 16a and 16b also preferably each have a volume that enable infusion source 14 to be exchanged, when near empty, without interrupting the surgical procedure. More specifically, chambers 16a and 16b preferably each have a volume of about 30 cc. Such volume allows about two minutes for a near empty infusion source 14 to be exchanged during conditions of maximum flow (e.g. core vitrectomy). In addition, once infusion source 14 is exchanged, all air bubbles within fluid lines 70, 72, and 74 will be automatically “scrubbed out” as the inactive chamber 16a or 16b refills, without the need for re-priming.

In the case of failure of either of chambers 16a or 16b, microprocessor 28 can preferably continue surgery with only one active chamber. In the case of failure of both chambers 16a and 16b, microprocessor 28 can preferably continue surgery using only infusion source 14.

A footswitch or foot controller 150 is preferably electrically coupled to microprocessor 28 via an interface 152. Footswitch 150 may be used to provide proportional control to a parameter of surgical device 29 via actuation of a pivotal treadle 154 via a surgeon's foot in the conventional manner. Exemplary surgical devices 29 utilized in anterior segment ophthalmic surgery include an irrigation/aspiration handpiece and an ultrasonic handpiece. A preferred ultrasonic handpiece is a phacoemulsification handpiece. Exemplary surgical devices utilized in posterior segment ophthalmic surgery include an extrusion handpiece and a victrectomy probe. When treadle 154 is in a depressed position, infusion fluid and ophthalmic tissue are typically aspirated via surgical device 29, and intraocular pressure is controlled as described hereinabove. When treadle 154 is in a fully undepressed position (or some other position) in which no aspiration is occurring at the surgical site, the control of intraocular pressure, as described hereinabove, is preferably ceased or deactivated. More specifically, if footswitch 150 signals microprocessor 28 that treadle 154 is in a fully undepressed position (or some other position) in which no aspiration is occurring at the surgical site, microprocessor 28 sends appropriate signals to close solenoid valve 44 and proportional solenoid valve 36 (if chamber 16b is active) or to close solenoid valve 46 and proportional solenoid valve 38 (if chamber 16a is active). In this manner, temporary, large increases in intraocular pressure, and unwanted flow of vitreous, infusion fluid, or other tissues from scleratomies, are avoided when surgical device 29 is not in use.

From the above, it may be appreciated that the present invention provides an improved method of controlling intraocular pressure with a microsurgical system. The present invention is illustrated herein by example, and various modifications may be made by a person of ordinary skill in the art. For example, while the present invention is described above relative to controlling intraocular pressure in an ophthalmic microsurgical system, it is also applicable to controlling pressure within the operative tissue during other types of microsurgery.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims

What is claimed is:

1. A method of controlling intraocular pressure with a microsurgical system, comprising the steps of:
providing an infusion chamber containing an irrigating fluid;
selecting a desired intraocular pressure;
pressurizing said infusion chamber with a pressurized gas to provide irrigating fluid to a surgical device;
aspirating a second fluid from an eye with said surgical device;
measuring a flow rate of said irrigating fluid within a fluid line fluidly coupled to said surgical device;
providing a signal corresponding to said measured flow rate to a computer;
calculating a predicted intraocular pressure with said computer in response to said signal;
adjusting a level of said pressurized gas in response to a second signal from said computer to maintain said predicted intraocular pressure proximate said desired intraocular pressure; and
ceasing said pressurizing step in response to said surgical device ceasing to aspirate said second fluid from said eye.

2. The method of claim 1 further comprising the steps of:
providing a footswitch having a pivotable treadle, wherein said treadle causes said surgical device to aspirate said second fluid from said eye in a first position, and wherein said treadle causes said surgical device to cease such aspiration in a second position; and
ceasing said pressuring step in response to said treadle being in said second position.

3. The method of claim 2 wherein said second position is a fully undepressed position of said treadle.
